(19) United States
(12) Patent Application Publication
(10) Pub. No.: US 2014/0303166 A1
(43) Pub. Date: Oct. 9, 2014

(54) COSMETIC METHOD FOR CHANGING THE APPEARANCE OF EYES

(71) Applicant: Gordon C. Tang, Bellevue, WA (US)

(72) Inventor: Gordon C. Tang, Bellevue, WA (US)

(21) Appl. No.: 13/858,866

(22) Filed: Apr. 8, 2013

(51) Int. Cl. A61K 31/382; A61K 31/5575; A61K 31/5377

(52) U.S. Cl. CPC A61K 31/382; A61K 31/5377; A61K 31/5575
USPC 514/236.2; 514/530; 514/432; 514/573; 514/622

(57) ABSTRACT

The current application is directed to cosmetic methods for changing the appearance of eyes by causing a retraction of the eyelids so that the eyes appear to be more open and have a wider appearance. Various methods involve application of one or more prostaglandins, synthetic prostaglandins, including synthetic prostaglandin F, and synthetic prostaglandin analogues, to eyelids, which results in contraction of eyelid muscles and a wider, more open appearance of the eyes.

### CROSS-REFERENCE TO RELATED APPLICATIONS

This application is a continuation-in-part of Application Ser. No. 13/567,931, filed Aug. 6, 2012, which claims the benefit of Provisional Application No. 61/515,815, filed Aug. 5, 2011.

### TECHNICAL FIELD

The present invention is related to cosmetic methods and, in particular, to a cosmetic method for changing the appearance of eyes.

### BACKGROUND

Drooping eyelids, a condition referred to as “ptosis,” is an often age-related condition that can also be caused by diseases and injuries or by congenital defects, can lead to an undesirable appearance as well as restricted vision. Often, an individual may not have ptosis, but may nonetheless desire, for appearance and cosmetic reasons, for his or her eyelids to appear to open more fully in order to enhance facial appearance. Currently, surgery is most commonly employed to treat ptosis and to cosmetically enhance eyelid appearance. Surgery is, however, expensive and surgery is, by no means, risk free.

### SUMMARY

The current application is directed to cosmetic methods for changing the appearance of eyes by causing a retraction of the eyelids so that the eyes appear to be more open and have a wider appearance. Various methods involve application of one or more prostaglandins, synthetic prostaglandins, including synthetic prostaglandin F, and synthetic prostaglandin analogues, to eyelids, which results in contraction of eyelid muscles and a wider, more open appearance of the eyes. The prostaglandins, synthetic prostaglandins, and synthetic prostaglandin analogues is, in certain of the cosmetic methods, mechanically applied to the eyelid and to surrounding tissue in a topical fashion, either in pure form or in solution or suspension within any of various different types of creams, ointments, solvents, or other appropriate vehicles for topical application. The prostaglandin, synthetic prostaglandin, and synthetic prostaglandin analogues is applied according to any of various different application schedules, including multiple times during the day, daily, multiple times during the week, weekly, or according to other schedules. The scheduled application is varied, in certain of the cosmetic methods, depending on whether or not the prostaglandin, synthetic prostaglandin, or synthetic prostaglandin analogue is topically applied in pure form or dissolved or suspended in any of various creams, ointments, or solutions. Additional cosmetic methods include injection of prostaglandin, synthetic prostaglandin, or synthetic prostaglandin analogues into eyelid tissues and surrounding tissues. Topical application may involve application by finger, by various types of applicators, by a spray applicator or nebulizer, and by patches, sleeping masks, and other such application means coated or otherwise treated with prostaglandin, synthetic prostaglandin, or synthetic prostaglandin analogue patches, sleeping masks, and other such application means. The prostaglandin, synthetic prostaglandin, and synthetic prostaglandin analogues, in certain cosmetic methods, is be included in eye shadow and other existing cosmetic solutions, powders, creams, and ointments.

### BRIEF DESCRIPTION OF THE DRAWINGS

FIG. 1 shows the structural formula for travoprost.

FIG. 2 shows the structural formula for dorzolamide hydrochloride.

FIG. 3 shows the structural formula for timolol maleate.

FIG. 4 shows the structural formula for unoprostone.

FIG. 5 shows the structural formula for latanoprost.

FIG. 6 shows the structural formula for bimatoprost.

### DETAILED DESCRIPTION

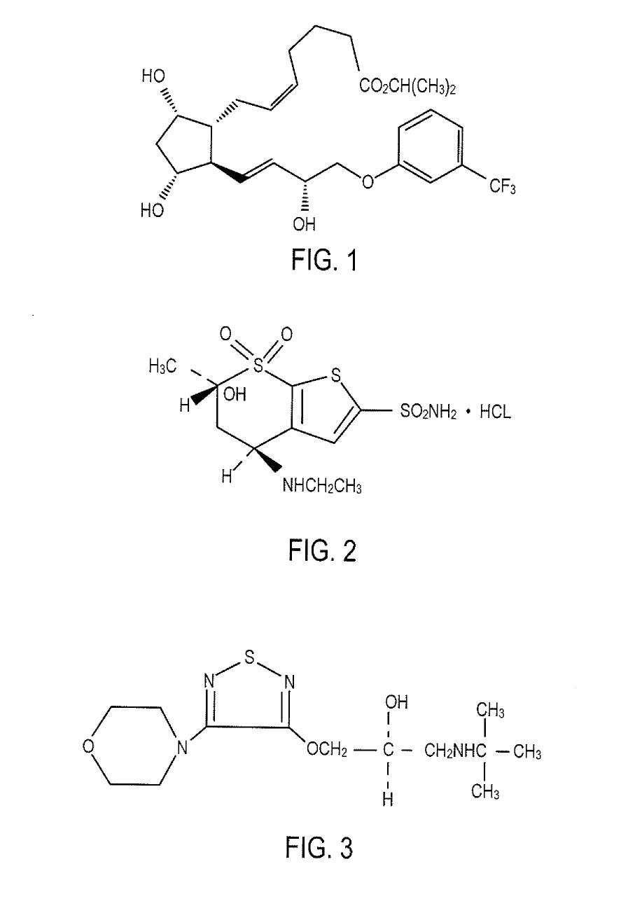

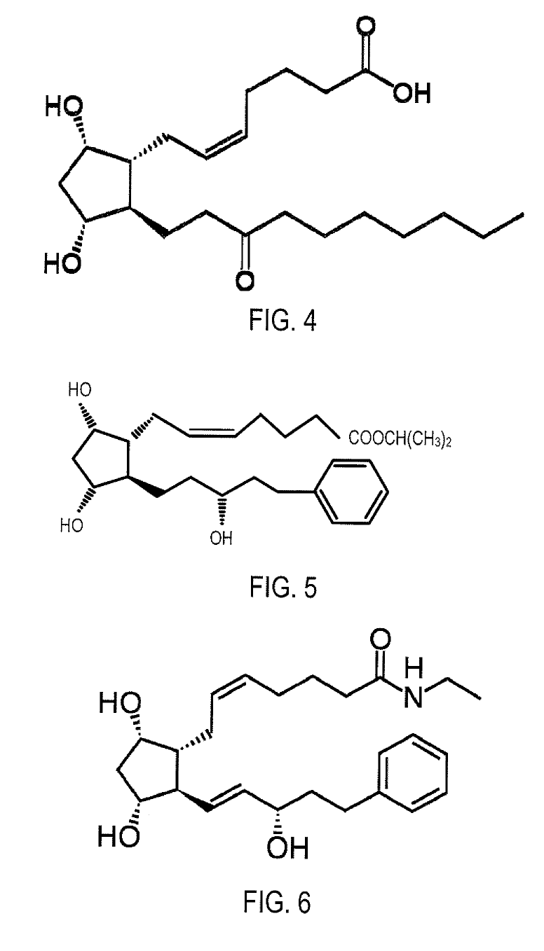

Many medications are currently used to treat glaucoma and ocular hypertension. These include: (1) Travatn®, which contains travoprost, propan-2-yl 7-[3,5-dihydroxy-2-[3-hydroxy-4-[3-(trifluoromethyl)phenoxy]-but-1-enyl]-cyclopentyl]hept-5-enoate, a synthetic prostaglandin F analogue, the structural formula for which is shown in FIG. 1; (2) Cosopt®, which includes dorzolamide hydrochloride,(4S,6S)-2-ethylamino-4-methyl-5,5-dioxo-5λ⁶,7dithiabicyclo[4.3.0] nona-8,10 -diene-8-sulfonamide, the structural formula for which is shown in FIG. 2; (3) timolol maleate, (S)-1-(tert-butylamino)-3-[(4-morpholin-4-yl-1,2,5-thiadiazol-3-yl)oxy]propan-2-ol, the structural formula for which is shown in FIG. 3; (4) Rescula®, which includes unoprostone isopropyl, isopropyl (Z)-7-[(1R,2R,3R,5 S)-3,5-dihydroxy-2-(3-oxodecyl)cyclopentyl] hept-5-enoic acid, the structural formula for which is shown in FIG. 4; (5) Xalatan®, which contains latanoprost, isopropyl (Z)-7-[(1R,2R,3R,5S)-3,5-dihydroxy-2-[(3R)3-hydroxy-5-phenylpentyl]-cyclopentyl] hept-5-enoate. the structural formula for which is shown in FIGS. 5; and (6) Lumigan®, which contains bimatoprost, 7-[3,5-dihydroxy-2-(3 -hydroxy-5-phenyl-pent-1-enyl)-cyclopentyl]-N-ethyl-hept-5-enamide, the structural formula for which is shown in FIG. 6. These compounds are normally suspended or dissolved in sterile, isotonic, buffered aqueous solutions for use as eye drops, and effect lowering of the internal pressure within the eye, or intraocular pressure.

Recently, the current inventor has discovered that travoprost, dorzolamide hydrochloride, timolol maleate, unoprostone, latanoprost, and bimatoprost can be topically applied, alone or in combination, to the eyelids in order to cause the eyelids to retract more completely, thus ameliorating ptosis and/or improving facial appearance. Topical treatments to which the current application is directed include ointments, creams, and solutions that contain one or more of the compounds travoprost, dorzolamide hydrochloride, and timolol maleate, alone or in combination, and/or one or more chemically-related compounds and may also include pure forms of various types of prostaglandin, synthetic prostaglandin, and synthetic prostaglandin analogues. These ointments and solutions are topically applied to eyelids in order to treat ptosis and/or to improve facial appearance. The ointments and solution may include, in addition to the above-mentioned active ingredients, stabilizers, buffering compounds, salts, carriers, viscosity enhancers, colorants, emollients, emulsifiers, bactericidal and germicidal agents, preservatives, and other ingredients that improve shelf life, application, consistency, and compatibility with topical application, among other things.

It turns out, as has been revealed by various experiments and trials, that application of pure or concentrated solutions of prostaglandin, synthetic prostaglandin, or synthetic prostaglandin analogues to eyelid tissue and/or surrounding tissues is effective in raising eyelids to give a more open, wider appearance to the eyes of a patient or user. Either pure prostaglandin, synthetic prostaglandin, or synthetic prostaglandin analogues may be rubbed into the eyelid tissues or a relatively concentrated solution of prostaglandin in any of various organic solvents, including ethanol, or ethanol-water mixtures, can be applied to produce the effect. Prostaglandin, synthetic prostaglandin, or synthetic prostaglandin analogs may be additionally included in various eye shadows and other cosmetic products that are routinely applied to eyelids in order to produce the eye-widening cosmetic effect, as well as in many different mixtures, suspensions, solutions, powders, ointments, creams, and other substances.

Any of a variety of different frequencies of application, total amount of prostaglandin topically applied during each application, and other such parameters of a cosmetic application schedule may be varied in accordance with the extent of the eye-widening effect desired, individual patients, and other such considerations. These considerations may, for example, include other types of cosmetics, treatment substances, and products routinely used by a patient, the overall condition of a patient's skin, the age of the patient, and other such considerations.

The methods to which the current application are directed include application of prostaglandin, synthetic prostaglandin, or synthetic prostaglandin analogues to eyelid tissue by finger, by any of various types of mechanical applicators, by spray application from a spray applicator, nebulizer, or atomizer, by dusting application, and by applying patches, eye masks, or other materials to the eyelids that are coated or infused with one or more prostaglandin, synthetic prostaglandin, or synthetic prostaglandin analogues.

1. Method for cosmetically changing the appearance of eyes, the method comprising:
loading an applicator with one or more prostaglandin, synthetic prostaglandin, or synthetic prostaglandin analogues; and
applying the prostaglandin, synthetic prostaglandin, or synthetic prostaglandin analogues to eyelids.

2. The method of claim 1 wherein the one or more prostaglandin, synthetic prostaglandin, or synthetic prostaglandin analogues includes:
travoprost;
dorzolamide hydrochloride;
timolol maleate;
unoprostone;
latanoprost; and
bimatoprost.

3. The method of claim 1 wherein the one or more prostaglandin, synthetic prostaglandin, or synthetic prostaglandin analogues is applied to the eyelids at a frequency selected from among:
multiple times each day;
daily;
multiple times each week;
weekly; and
multiple times a month.

4. The method of claim I wherein the one or more prostaglandin, synthetic prostaglandin, or synthetic prostaglandin analogues is applied in pure form.

5. The method of claim 1 wherein the one or more prostaglandin, synthetic prostaglandin, or synthetic prostaglandin analogues is dissolved in a solvent or solution.

6. The method of claim 1 wherein the one or more prostaglandin, synthetic prostaglandin, or synthetic prostaglandin analogues is suspended in a solvent or solution.

7. The method of claim 1 wherein the one or more prostaglandin, synthetic prostaglandin, or synthetic prostaglandin analogues is included in a mixture.

8. The method of claim I wherein the one or more prostaglandin, synthetic prostaglandin, or synthetic prostaglandin analogues is included in one or more of:
a cream;
an ointment; and
a powdered cosmetic.
